(19) United States
(12) Patent Application Publication
(10) Pub. No.: US 2003/0158493 A1
(43) Pub. Date: Aug. 21, 2003

(54) Method for determining hemodynamic state

(76) Inventors: Daniel Goor, Tel Aviv (IL); Gad Cotter, Tel Aviv (IL); Yaron Moshkovitz, Tel Aviv (IL)

Correspondence Address: GREER, BURNS & CRAIN, 300 S WACKER DR, 25TH FLOOR, CHICAGO, IL, 60606, US

(21) Appl. No.: 10/221,764

(22) PCT Filed: Mar. 12, 2001
(86) PCT No.: PCT/IL01/00234

(30) Foreign Application Priority Data
Mar. 13, 2000 (IL) 135032

(51) Int. Cl. A61B 5/04

(52) U.S. Cl. 600/513

(57) ABSTRACT

A method for determining the hemodynamic state of a subject. The method comprises (a) determining the cardiac power index (Cpi) and systemic vascular resistance index (SVRi) values of a plurality of patients who have been diagnosed as having a specified hemodynamic state; (b) determining the range of Cpi and SVRi paired values corresponding to each of the hemodynamic states; (c) determining the Cpi and SVRi paired value of the subject; (d) comparing the Cpi and SVRi paired value of the subject to the ranges of Cpi and SVRi paired values determined in step (b); and (e) determining the range of Cpi and SVRi paired values which is most similar to the Cpi and SVRi paired value of the subject. The hemodynamic state which corresponds to the range indicates the hemodynamic state of the subject.

### FIELD OF THE INVENTION

This invention relates to the determination of the hemodynamic state of a patient by use of parameters of cardiac and peripheral vascular performance.

### BACKGROUND OF THE INVENTION

The following references may be relevant to the understanding of the invention, and are referred to in the specification by number:

1. Roul G, Moulichon M. E., Bareiss P, Gries P, Koegler A, Sacrez J, Germain P, Mossard J. M., Sacrez A, Prognostic factors of chronic heart failure in NYHA class II or III: value of invasive exercise haemodynamic data. Eur Heart J (1995); 16:1387-98.

2. Marmor A, Schneeweiss A. Prognostic value of noninvasively obtained left ventricular contractile reserve in patients with severe heart failure. J Am Coll Cardiol (1997) Feb;29(2):422-8.

3. Marmor A, Jain D, Cohen L S, Nevo E, Wackers F J, Zaret B L. Left ventricular peak power during exercise: a noninvasive approach for assessment of contractile reserve. J Nucl Med (1993) Nov;34(11):1877-85.

4. Tan L B. Cardiac pumping capability and prognosis in heart failure. Lancet (1986) 13(2):1360-63.

5. Sharir T, Feldman M D, Haber H, Feldman A M, Marmor A, Becker L C, Kass D A. Ventricular systolic assessment in patients with dilated cardiomyopathy by preload-adjusted maximal power—Validation and noninvasive application. Circulation (1994) May;89(5):2045-53.

6. Tan L B. Clinical and research implications of new concepts in the assessment of cardiac pumping performance in heart failure. Cardiovasc Res (1987) Aug;21(8):615-22.

7. Cotter G, Metzkor E, Kaluski E, Faigenberg Z, Miller R, Simovitz A, Shaham O, Margithay D, Koren D, Blatt A, Moshkovitz Y, Zaidenstein R, Golik A. Randomized trial of high-dose Isosorbide Dinitiate plus low-dose Furosamide versus high-dose Furosamide plus low-dose Isosorbide Dinitrate in severe pulmonary oedema. Lancet. (1998); 351: 389-93.

8. Cotter G, Kaluski E, Blatt A, Milovanov O, Moshkovitz Y, Zaidenstein R. Salah A, Alon D, Mihovitz Y, Metzger M, Vered Z, Golik A. L-NMMA (a Nitric Oxide Synthase Inhibitor) is Effective in the Treatment of Cardiogenic Shock. Circulation. 2000 Mar 28;101(12):1358-61.

9. P. D. Sasieni, Statistical Analysis of the performance of diagnostic tests (Invited review), Cytopathology, 1999, 10,73-78.

10. Jeroen G. Lijmer, Ben Willen Mol,Siem Heisterkamp, Gouke J. Bonsel, Martin H. Prins, Jan H. P., van der Meulen, Patrik M. M. Bossuyt. Empirical Evidence of Design Related Bias in Studies of Diagnostic Tests, JAMA, 1999, 282,11,1061-1066.

11. SAS/STAT User's Guide, Version 6, Fourth Edition. Volume 1, Cary, N.C.:SAS Institute Inc., 1989.

To date, no correlation has been found between invasive hemodynamic measurements and the clinical syndrome of patients with congestive heart failure (CHF) (1). In patients admitted with acute deterioration in cardiac function such as progressive dyspnea leading to pulmonary edema or cardiogenic shock, and even in patients with systolic chronic stable CHF, the measurement of cardiac index (CI) or systemic vascular resistance index (SVRiᵢ) has not provided any reliable diagnostic, therapeutic or prognostic value.

SVRᵢ is a measure of the resistance of the vascular system to blood flow and is measured in Kg. * M⁴/sec³ (=wood*M²). In the cardiovascular system, SVR_{I}=(mean arterial blood pressure (MAP)−right arterial pressure)/CI. If not obtainable, right arterial pressure may be estimated as 10-15% of MAP.

Cardiac power index (Cpᵢ) is a measure of the contractile state of the myocardium and is measured in watts/M². The measurement of Cpᵢ is a newly introduced concept in cardiology (2-6). It is based on the physical law of fluids where

Power=Flow X Pressure.

In the cardiovascular system, Cpᵢ can be measured by replacing flow with cardiac index (CI) and pressure by the MAP.

Therefore:

Cpᵢ=CI X MAP.

This measurement was partially used in the past (2-6) to evaluate the cardiac contractility of patients with CHF. It may be assumed that in patients with CHF, as Cpᵢ progressively decreases a compensatory increase of SVRᵢ occurs, and this increase is predictable within normal ranges. In addition, in patients with acute decrease in Cpᵢ this SVRᵢ response could be either (1) adequate—leading to a compensated or near compensated response, (2) excessive—leading to a significantly higher than required MAP increase, thereby leading to pulmonary edema, or (3) insufficient—leading to low MAP, inadequate perfusion of vital organs (brain, heart, kidneys) and cardiogenic shock.

### SUMMARY OF THE INVENTION

It is an object of the present invention to provide a method for determining the hemodynamic state of a patient.

It is a further object of the invention to provide a method for monitoring changes in the hemodynamic state of a patient.

Thus, the present invention provides a method for determining the hemodynamic state of a subject comprising:

(a) determining the cardiac power index (Cpᵢ) and systemic vascular resistance index (SVRᵢ) values of a plurality of patients who have been diagnosed as having a hemodynamic state selected from the group consisting of systolic congestive heart failure (sCHF), pulmonary edema (PE), cardiogenic shock (CS), vasodilative shock (VS) and normal state;

(b) determining the range of Cpᵢ and SVRᵢ paired values corresponding to each of said hemodynamic states;

(c) determining the Cpᵢ and SVRᵢ paired value of said subject;

(d) comparing the Cpᵢ and SVRᵢ paired value of said subject to the ranges of Cpᵢ and SVRᵢ paired values determined in step (b); and

(e) determining the range of Cpᵢ and SVRᵢ paired values which is most similar to the Cpᵢ and SVRᵢ paired value of said subject, the hemodynamic state corresponding to said range indicating the hemodynamic state of said subject.

It has now been surprisingly found that for a given patient, the values of the pair of parameters Cpᵢ and SVRᵢ are indicative of the hemodynamic state of the patient. In this specification, the term “paired values” will be used to indicate the Cpᵢ and SVRᵢ values of a given patient measured at essentially the same time.

The method of the present invention enables the determination of the hemodynamic state of a patient by determining only two parameters, Cpᵢ and SVRᵢ. These parameters may be determined either invasively, e.g. with a Swan-Ganz catheter or arterial line, or non-invasively, e.g. by Echo-doppler or non-invasive blood pressure measurement. The obtained values are then compared to a set of values previously compiled from patients with known hemodynamic states. The comparison may be carried out graphically, by eye, or by calculation (e.g. by computer). The range of Cpᵢ and SVRᵢ paired values which is most similar to the Cpᵢ and SVRᵢ paired value of said subject will indicate in which group the subject should be classified. Similarity may be determined by eye (for example when using a graph) or by known statistical methods.

The known hemodynamic states used in the method of the invention are: (1) systolic or compensated CHF (sCHF). This group also includes hypertensive patients (HTN), due to their similar hemodynamic profile and small number in the study; (2) PE; (3) CS; (4) vasodilative or septic shock (VS); and (5) a group termed “normal” which represents patients who do not suffer from CHF. The last group consists of normal patients, i.e. with an SVRᵢ of approximately 15-35 wood*M² and a Cpᵢ above 190 watt/M².

The position of the patient's paired Cpᵢ and SVRᵢ values provide an indication as to how to treat the patient. For example, if the paired values are located in the range of values typical of cardiogenic shock, it would be advisable to administer to the patient a treatment which will boost vascular resistance (8). On the other hand, if the paired values are located in the range of values typical for pulmonary edema, it would be advisable to administer to the patient a treatment which will decrease vascular resistance (7).

Changes in the condition of the patient due either to the natural progression of the disease or to therapeutic treatment, may be easily monitored using the method of the invention by following the change in position of the paired Cpᵢ and SVRᵢ values of the patient with respect to the predetermined set of values. In this way, the effectivity of a treatment may be assessed. Thus, the method of the invention may have significant therapeutic implications through pharmaceutical manipulation of SVRᵢ by vasodilators (nitrates, endothelin antagonists) or vasoconstrictors (L-NMMA, vasopresin).

A graph prepared according to the method of the invention may appear, for example, on the display of a monitor, so that the measured Cpᵢ and SVRᵢ values of a patient can be immediately plotted on the graph in order to determine the patient's “real time” condition.

### BRIEF DESCRIPTION OF THE DRAWINGS

In order to understand the invention and to see how it may be carried out in practice, a preferred embodiment will now be described, by way of non-limiting example only, with reference to the accompanying drawings, in which:

FIG. 1 shows CI (litter/minute/M²) in the six following diagnosed groups: CS, PE, HTN, sCHF, normal and VS;

FIG. 2 shows Pulmonary Capillary wedge pressure (mmHg) in the 6 groups;

FIG. 3 shows Cpii (watt/M²) in the 6 groups;

FIG. 4 shows SVRii (wood*M²) in the 6 groups; and

FIG. 5 is a graph in which the Y-axis indicates Cpᵢ units (in watts/M²) and the X-axis indicates SVRᵢ units (Wood*M² units). The graph (also termed in this specification a “nomogram”) is used for classification of the hemodynamic status of patients and may be constructed by a method of statistical analysis according to one embodiment of the method of the invention. Normal patients are indicated by (Δ), PE patients are indicated by (□), CS patients are indicated by (O), VS patients are indicated by (*) and sCHF and HTN patients are indicated by (●).

### DETAILED DESCRIPTION OF PREFERRED EMBODIMENTS

### Example 1

Determination of hemodynamic state by graphic means

Patients and Methods.

Hemodynamic data was obtained in patients undergoing right heart catheterization.

Inclusion Criteria:

All patients who were diagnosed by conventional clinical criteria (see below) as having systolic CHF (sCHF), hypertensive crisis, acute pulmonary edema (PE), vasodilative shock or cardiogenic shock were included.

Exclusion Criteria:

Significant valvular disease, significant brady- or tachy-arrhythmias or renal failure (creatinine>2.5 mg/dl).

Clinical Diagnosis Criteria:

1) Systolic CHF: Patients admitted for invasive hemodynamic assesment due to CHF exacerbation, defined as clinical symptoms and signs of CHF, NYHA class III-IV, accompanied by EF<35% on echocardiography and not treated with any oral drugs for 6 hours or intravenous drugs for the last 2 hours; not fulfilling the criteria for cardiogenic shock or pulmonary edema.

2) Pulmonary edema: patients admitted due to clinical symptoms and signs of acute pulmonary congestion accompanied by findings of lung edema on chest X-Ray and O₂ saturation<90% on room air by pulse oxymetery during invasive measurements.

3) Cardiogenic shock: Systolic blood pressure<100 mmHg for at least one hour after percutaneous revascularization due to an acute major coronary syndrome not responsive to revascularization, mechanical ventilation, Intra-Aortic Balloon-Pump (IABP), IV fluids administration and dopamine of at least 10 μg/kg/min and accompanied by signs of end organ hypoperfusion but not accompanied by fever>38° or a systemic inflammatory syndrome.

4) Vasodilative shock: Systolic blood pressure<100 mmHg accompanied by fever>38°, systemic inflammatory syndrome and signs of end organ hypoperfusion for at least 3 hours not responsive to IV fluids and IV dopamine of at least 10 μg/kg/min.

5) Hypertension: MAP>135 mmHg without signs of end-organ hypoperfusion, ischemia or pulmonary edema These patients were included in the sCHF group.

Hemodynamic Variables assesment:

In all patients the hemodynamic variables were obtained during right heart catheterization using a Swan-Ganz cathteter placed under fluroscopic guidence. All measurments were obtained while patients were at least 30 seconds without IABP while on the same treatment used at the time the clinical diagnosis was made.

CI was measured by thermodilution using the mean of at least 3 consecutive measurements within a range of <15%. In Normal subjects, right heart catheterization was not performed due to ethical concerns. The values used in this cohort were obtained by standard non-invasive cuff blood pressure measurement and evaluation of CI by the FDA-approved NICaS 2001, a non-invasive on-line cardiac output monitor (Cohen J A, Arnaudov D, Zabeeda D, Schlthes L, Lashinger J, Schachner A. Non-invasive measurment of cardiac output during coronary artery bypass grafting. Eur. J. Card. Thoracic Surg. 1998; 14: 64-9). Therefore, wedge pressure was not assessed in normal subjects. Instead, we used standard values documented in the litterature (Lange R A, Hillis L D. Cardiac catheterization and hemodynamic assessment. In: Topol E J; Textbook of Cardiovacular Medicine).

Hemodynamic variables calculation:

Cpᵢ was determined as MAP×CI and SVRᵢ was determined as (MAP−right atrial pressure)/CI. As right atrial pressure was not measured in normal subjects, it was estimated to be 10% of MAP.

Results:

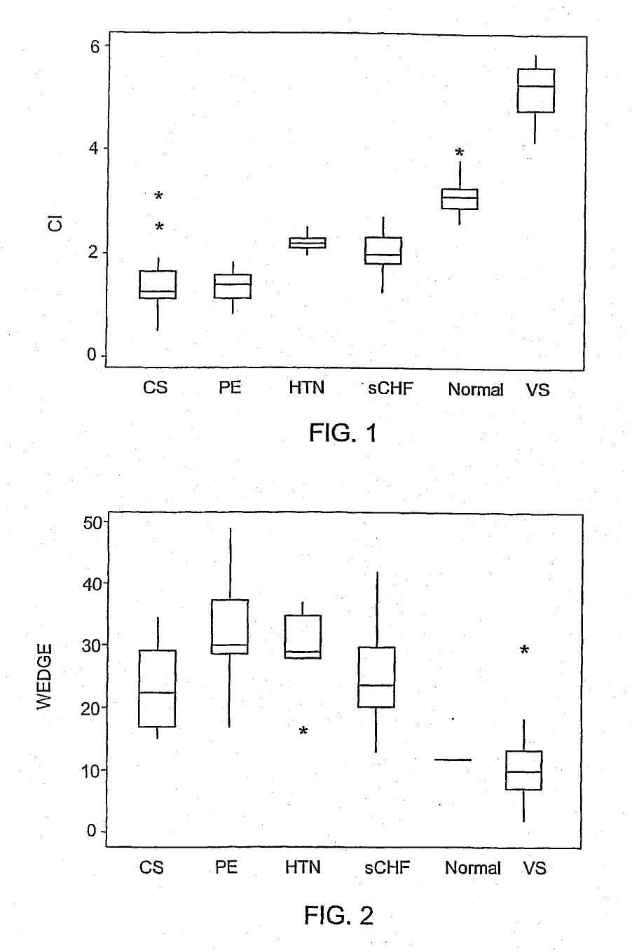

One hundred consecutive patients (56 patients with systolic CHF, 5 patients with HTN crisis, 11 patients with pulmonary edema, 17 patients with cardiogenic shock and 11 patients with vasodilative shock) and twenty healthy volunteers were enrolled in the study. The mean CI, wedge pressure, MAP, SVRᵢ and Cpᵢ according to clinical diagnosis are presented in Table 1 and as box-plots in FIGS. 1-4. Since the number of patients with hypertensive crisis (HTN) was too small to yield a statisticaly meaningful analysis, they were incorporated into the systolic CHF group for all further analysis.

TABLE 1
The means and standard deviations of various parameters in
the 5 diagnosis groups

| GROUP | No. Obs. | Variable | Mean | Std. Dev. |
|---|---|---|---|---|
| CHF | 61 | SVRiI | 44.8666667 | 8.0327015 |
| | | CPI | 210.6833333 | 60.1848823 |
| | | WEDGE | 25.5166667 | 7.1556347 |
| | | MAP | 101.1833333 | 17.9806786 |
| | | CI | 2.0611667 | 0.3313153 |
| Pulmonary | 11 | CVRI | 88.1818182 | 16.7380894 |
| Edema | | CPI | 182.2727273 | 57.3673965 |
| | | WEDGE | 32.7272727 | 8.6033820 |
| | | MAP | 131.3636364 | 12.6828445 |
| | | CI | 1.3727273 | 0.3196589 |
| Normal | 20 | SVRiI | 25.1500000 | 4.0817308 |
| | | CPI | 280.0000000 | 35.7402913 |
| | | WEDGE | — | — |
| | | MAP | 87.9000000 | 8.8549718 |
| | | CI | 3.2000000 | 0.3568871 |
| Septic Shock | 11 | SVRiI | 11.8181818 | 1.1241158 |
| | | CPI | 358.1818182 | 56.4921555 |
| | | WEDGE | 11.3636364 | 7.6976974 |
| | | MAP | 68.1818182 | 5.4372453 |
| | | CI | 5.2181818 | 0.5344496 |
| Cardiogenic | 17 | SVRiI | 55.6375000 | 31.0761833 |
| Shock | | CPI | 98.9375000 | 34.9866046 |
| | | WEDGE | 23.3125000 | 6.5086481 |
| | | MAP | 72.1875000 | 11.2973079 |
| | | CI | 1.4218750 | 0.6426427 |

Hemodynamic Variables:

1) Cardiac Index (CI) (FIG. 1): The mean values of CI were significantly lower in patients with systolic CHF, pulmonary edema and cardiogenic shock compared to normals and higher in patients with vasodilative shock. ROC analysis found the cut-off point of CI<2.7 Lit./min./M² useful for the determination that a patient has any kind of heart failure (either systolic CHF, pulmonary edema or cardiogenic shock)(sensitivity=1, specificity=0.99). However, values between 1.2-2.7 Lit./min./M² could be found in all patients with systolic CHF, 73% of patients with pulmonary edema and 47% of patients with cardiogenic shock. Moreover, the mean CI of patients in pulmonary edema and cardiogenic shock was found to be almost identical (1.4±0.4 vs 1.35±0.7 L/min/M², p=ns).

2) Mean Arterial Blood Pressure (MAP): As compared to normals, the mean values of MAP were significantly higher in patients with pulmonary edema and by definition, higher in patients with HTN crisis and lower in vasodilative and cardiogenic shock. Despite this, large areas of overlap were found regarding MAP measurments between pulmonary edema, systolic CHF and HTN crisis (MAP>100 mmHg) and between systolic CHF, cardiogenic shock and vasodilative shock (MAP<100 mmHg).

3) Pulmonary capillary wedge pressure (FIG. 2): As compared to normals, the mean wedge pressure was significantly higher in patients with systolic CHF and pulmonary edema and lower in patients with vasodilative shock. The analysis was based on the normal values for wedge pressure reported in the literature (<12 mmHg (8))(p=0.001). However, the overlap of wedge pressure values among the groups was very extensive. Values between 12-38 mmHg were found in 82% of patients with systolic CHF, 64% of patients with pulmonary edema, 76% of patients with cardiogenic shock, and 18% of patients with vasodilative shock.

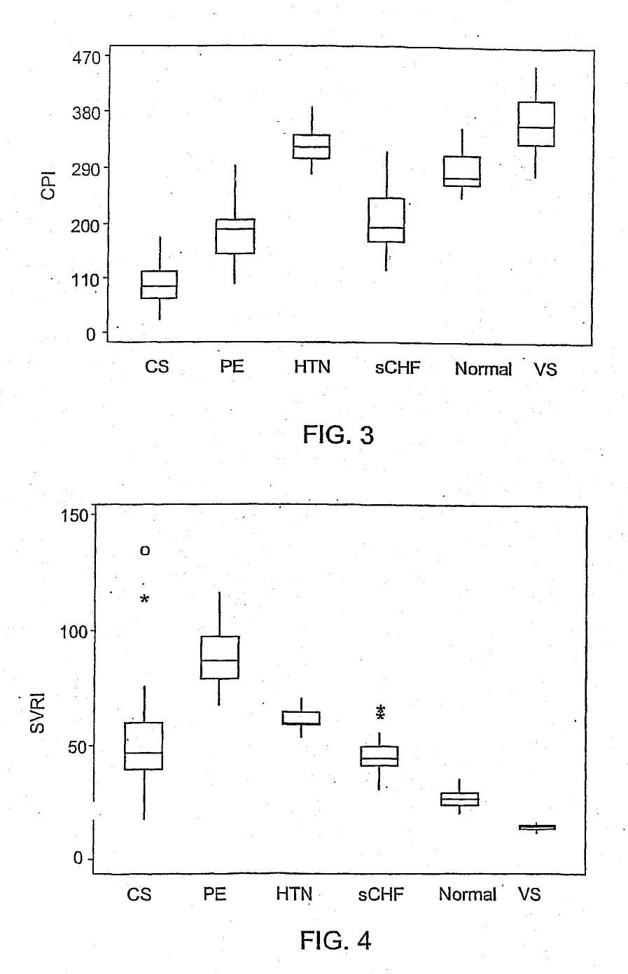

4) Cardiac Power index (FIG. 3): As compared to normals, the mean values of Cpᵢ were low in patients with systolic CHF and pulmonary edema, extremely low in patients with cardiogenic shock and high in patients with HTN crisis and vasodilative shock. However, some overlap was encountered among the 5 groups. Values of 200 to 300 Watt/M² were measured in 75% of normal people, 39% of patients with systolic CHF, 27% of patients with pulmonary edema, 18% of patients with vasodilative shock but none of the patients with cardiogenic shock (in whom Cpi was consistently below 170 Watt/M².

5) Systemic Vascular Resistence Index (FIG. 4): As compared to normals, the mean values of SVRᵢ were significantly higher in patients with systolic CHF and HTN crisis, extremely high in patients with pulmonary edema and lower in patients with vasodilative shock. ROC analysis found the cut-off point of SVRᵢ<35 wood*M² to be useful in discriminating normal subjects from patients with any CHF syndrome (specificity=1, sensitivity=0.95). Also, SVRᵢ was found instrumental in the diagnosis of pulmonary edema: all patients with this clinical syndrome had SVRᵢ>67 wood*M² while SVRᵢ values in all other patients as well as normal subjects were significantly lower than this value.

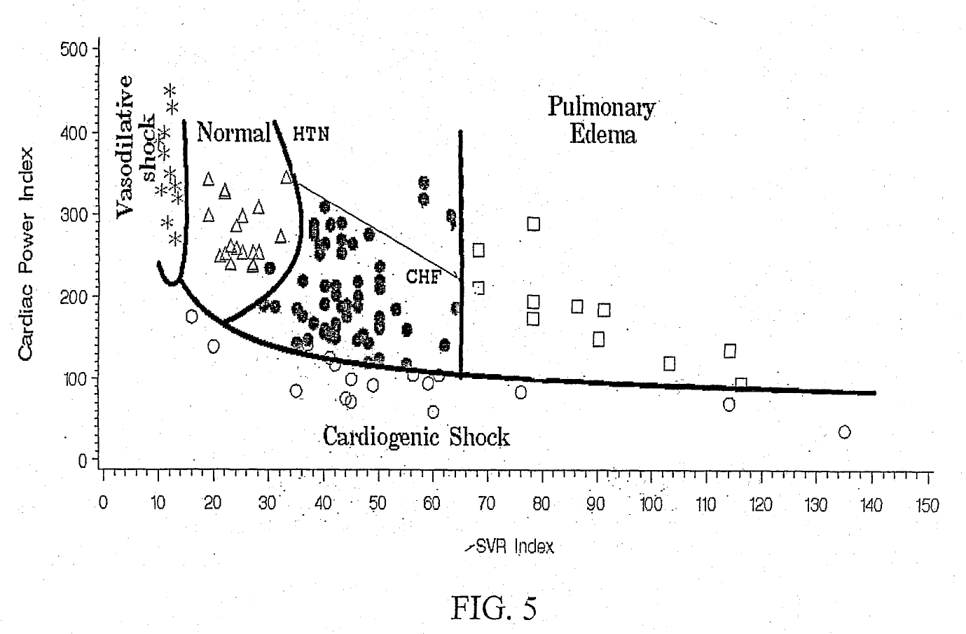

Cpi/SVRi graph (FIG. 5):

Distributions of SVRᵢ and Cpᵢ were highly skewed, whereas log(SVRᵢ) and Log(CPᵢ) were less skewed. Therefore, for her analysis only Log of the indices was used. However, the graph was constructed using values translated back from the Log values.

The distributions of the two log-parameters were different between groups. However, neither of the individual parameters enabled separation among the five groups, as shown in Table 2.

TABLE 2
Number of Observations Classified into the Correct Clinical Group
Using Log(Cpi) or Log(SVRi) only.

| | | | | | | |
|---|---|---|---|---|---|---|
| | Cardio- | | | | | |
| By Clinical | genic | Systolic | | Pulmonary | Septic | |
| diagnosis→ | Shock | CHF | Normal | Edema | Shock | Total |

(1) Classification using Log(CPi) only.
By
Parameters↓

| | | | | | | |
|---|---|---|---|---|---|---|
| Cardiogenic | 13 | 4 | 0 | 0 | 0 | 17 |
| Shock | | | | | | |
| Systolic CHF | 1 | 44 | 14 | 0 | 2 | 61 |
| Normal | 0 | 9 | 8 | 0 | 3 | 20 |
| Pulmonary | 1 | 9 | 1 | 0 | 0 | 11 |
| Edema | | | | | | |
| Septic Shock | 0 | 0 | 3 | 0 | 8 | 11 |

(2) Classification using Log(SVIRi) only.
By
Parameters↓

| | | | | | | |
|---|---|---|---|---|---|---|
| Cardiogenic | 2 | 12 | 1 | 2 | 0 | 17 |
| Shock | | | | | | |
| Systolic CHF | 0 | 58 | 3 | 0 | 0 | 61 |
| Normal | 0 | 3 | 17 | 0 | 0 | 20 |
| Pulmonary | 2 | 0 | 0 | 9 | 0 | 11 |
| Edema | | | | | | |
| Septic Shock | 0 | 0 | 0 | 0 | 11 | 11 |

These data suggested that the separation may be obtained using two dimensional discriminant analysis. We used classical discriminant analysis for Normal distributions with unequal covariance matrices because the small numbers of observations in two groups prevented from using more flexible kernel functions.

Due to large variability of variances of the parameters in the five groups, we could not suppose equal covariance matrices in the groups. (The test of homogeneity of within covariance matrices gives P<0.0001).

Classification using the nomogram.

In order to determine the state of a patient, his Cpᵢ and SVRᵢ are determined, and the paired values are plotted on a graph, e.g. FIG. 5. The location of the measured paired values on the graph indicates which clinical condition may be assigned to the patient.

The vascular response to decreased cardiac performance is crucial in determining the clinical syndrome of CHF. Insufficient SVRi increase may cause cardiogenic shock while excessive vasoconstriction will induce progressive pulmonary congestion resulting in frank pulmonary edema. The exact mechanism of deterioration of each patient can be determined using measurements of CI and MAP and a simple nomogram. This can have extensive therapeutic implication through pharmaceutical manipulation of SVRi. For example, ISDN can be used to move patients from PE to cCHF, and 1-NMMA can be used to move patients from cardiogenic shock.

### Example II

Determination of hemodynamic state using statistical analysis

Another embodiment of the method of the invention will be illustrated by means of the example given below. However, it will be clear to the skilled man of the art that other embodiments using other statistical methods of analysis are possible.

1. Data

Statistical Methods:

The five clinical groups were compared with regard to all parameters using a one-way Analysis of Variance. The Ryan-Einot-Gabriel-Welsch Multiple Range Test was used for pair-wise comparisons between the groups, while Dunnett's T test was used to compare all groups to the healthy controls.

A one-sample t-test was performed to compare mean Wedge pressure in each group to the wedge pressure of normal people (less than 12 mmHg).

In order to determine the usefulness of the hemodynamic parameters to discriminate between the clinical syndromes, ROC curves, derived from a Logistic regression model were applied to the data to determine the best cutoff point of various parameters in terms of highest sensitivity and specificity.

Cpi/SVRi normogram:

A classification rule was developed using second order discriminant analysis. Firstly both variables (CPᵢ and SVRᵢ) were transformed into Log scale for better approximation to normality. Since the number of patients with HTN was small, they were incorporated into the systolic CHF group. The classification used two steps. In the first step the rule separated three classes: Vasodilative shock Cardiogenic shock and combined group, which includes Normal patients, systolic CHF and Pulmonary Edema (N-C-P). If after the first step the patient was defined as N-C-P, the second classification was used for separation among Normal, Systolic CHF and Pulmonary Edema subgroups.

All calculations were performed by SAS 6.12 [SAS Institute Inc., Cary, N.C.] using procedures FREQ, MANS, GLM, DISCRIM, GPLOT.

2. Classification rule.

A. Classification using calculations.

Step 1. Calculate three values v1, v2, v3 according to the formulas below.

v1=LCPi2*21.54+2*LCPi*LSVRi*10.61+LSVRi2*59.44−LCPi*305.24−LSVRi*417.70+1408.89

v2=LCPi²*10.12+2*LCPi*LSVRi*5.67−LSVRi2*4.99−LCPi*135.81−LSVRi*90.11+482.61

v3=LCPi2*7.29+LCPi*LSVRi*2.57+LSVRi2*4.09−LCPi* 97.41−LSVRi*58.22+368.16

Classify the patient

into the group ‘Vasodilative shock’, if v1 is the smallest value

into the group ‘Cardiogenic Shock’, if v2 is the smallest value

if v3 is the smallest value go to step 2

Step 2. Calculate three values v4, v5, v6 according to the formula below.

v4=LCPi2*6.45−2*LCPi*LSVRi*0.45+LSVRi2*16.01−LCPi*65.16−LSVRi* 116.53+391.67

v5=LCPi2*17.75+2*LCPi*LSVRi*26.56+LSVRi2*54.27−LCPi*420.26−SVRi*758.55+2775.78

v6=LCPi2*32.95+2*LCPi*LSVRi*3.09+LSVRi2*19.72−LCPi*390.74−LSVRi*161.49+1355.57

Classify the patient

into the group ‘Systolic CHF’, if v4 is the smallest value among v4, v5, v6 and LSVRi<Log(67)

into the group ‘Pulmonary Edema’, if v5 is the smallest value among v4, v5, v6 and LSVRi>Log(67)

into the group ‘Normal’, if v6 is the smallest value among v4, v5, v6

The value of SVRi=67 was used to separate patients with systolic CHF from patients with pulmonary edema since the group of ‘pulmonary edema’ was rather small and by classifying these patients according to the usual rule we did not receive a separating line for Cpi measures>250 Watt/M². Therefore, the line of SVRi=67 wood*M² was used as an approximation of the classification results.

3. Classification results.

The results of the application of the classification rule to the sample are presented in Table 3.

TABLE 3
Number of Observations Classified into the Correct
Clinical Group using both Log(SVRᵢ) and Log(CPᵢ).

| | Cardio- | | | | | |
|---|---|---|---|---|---|---|
| By Clinical | genic | Systolic | | Pulmonary | Septic | |
| diagnosis→ | Shock | CHF | Normal | Edema | Shock | Total |
| By | | | | | | |
| Parameters↓ | | | | | | |
| Cardiogenic | 15 | 2 | 0 | 0 | 0 | 17 |
| Shock | | | | | | |
| Systolic CHF | 0 | 60 | 1 | 0 | 0 | 61 |
| Normal | 0 | 0 | 20 | 0 | 0 | 20 |
| Pulmonary | 2 | 0 | 0 | 11 | 0 | 11 |
| Edema | | | | | | |
| Septic Shock | 0 | 0 | 0 | 0 | 11 | 11 |

4. Performance of the classification rule.

The performance of the diagnostic procedure with only two possible results and two classes of patients usually is expressed by using measures like positive (negative) predictive value (9) or diagnostic odds ratio(10). For more complex tests with many outcomes and many classes of patients the overall performance may be expressed through the difference between proportion of erroneously classified patients with and without using the test. This measure is usually called as Lambda assymmetric (R|C), where R (rows) is the true group and C (column) is a group where the patient was classified. For our data, Lambda (R|C)=0.95 (S.D.(Lambda)=0.03) which corresponds to the 3 errors of classification according to the classification rule, instead of 59 errors of classification according to the prior probabilities of the groups.

1. A method for determining the hemodynamic state of a subject comprising:
(a) determining the cardiac power index (Cpᵢ) and systemic vascular resistance index (SVRᵢ) values of a plurality of patients who have been diagnosed as having a hemodynamic state selected from the group consisting of systolic congestive heart failure (sCHF), pulmonary edema (PE), cardiogenic shock (CS), vasodilative shock (VS) and normal state;
(b) determining the range of Cpᵢ and SVRᵢ paired values corresponding to each of said hemodynamic states;
(c) determining the Cpᵢ and SVRᵢ paired value of said subject;
(d) comparing the Cpᵢ and SVRᵢ paired value of said subject to the ranges of Cpᵢ and SVRᵢ paired values determined in step (b); and
(e) determining the range of Cpᵢ and SVRᵢ paired values which is most similar to the Cpᵢ and SVRᵢ paired value of said subject, the hemodynamic state corresponding to said range indicating the hemodynamic state of said subject.

2. A method according to claim 1 wherein said Cpᵢ and SVRᵢ paired values are plotted on a graph, and said range of Cpᵢ and SVRᵢ paired values indicative of each of said hemodynamic states is indicated by a delineated area on said graph.

3. A method according to claim 2 wherein said graph is substantially equivalent to FIG. 5.

4. A method according to claim 1 wherein said ranges of Cpᵢ and SVRᵢ paired values indicative of each of said hemodynamic states are calculated by statistical analysis and said Cpᵢ and SVRᵢ values of said subject are compared to said ranges by a statistical method.

5. A method according to claim 4 wherein said range of Cpᵢ and SVRᵢ paired values indicative of each of said hemodynamic states is displayed in a graph format on a display screen.

6. A method according to claim 1 wherein Cpᵢ is calculated from the product of the cardiac index (CI) and the mean arterial blood pressure (MAP).

7. A method according to claim 6 wherein said cardiac index and/or said blood pressure is measured by an invasive measuring technique.

8. A method according to claim 7 wherein said measuring technique for measuring the cardiac output employs a Swan-Ganz catheter.

9. A method according to claim 1 wherein cardiac output and/or said blood pressure is measured by a non-invasive measuring technique.

10. A method of monitoring the hemodynamic state of a subject, comprising:
(a) determining the Cpᵢ and SVRᵢ of said subject;
(b) determining the hemodynamic state of said subject by the method of claim 1;
(c) redetermining the Cpᵢ and SVRᵢ of said subject after a predetermined time;
(d) redetermining the hemodynamic state of said subject by the method of claim 1; and
(e) comparing the hemodynamic state obtained in steps (b) and (d).

11. A method of assessing the effect of a medical treatment on the hemodynamic state of a subject, comprising:
(a) determining the Cpᵢ and SVRᵢ of said subject;
(b) determining the hemodynamic state of said subject by the method of claim 1;
(c) administering said medical treatment to said subject;
(d) determining the Cpᵢ and SVRᵢ of said subject after said treatment;
(e) determining the hemodynamic state of said subject by the method of claim 1; and
(f) comparing the hemodynamic state obtained in steps (b) and (e).

12. An apparatus for determining the hemodynamic state of a subject, comprising:
(a) means for determining the cardiac power index (Cpᵢ) and systemic vascular resistance index (SVRᵢ) values of a plurality of patients who have been diagnosed as having a hemodynamic state selected from the group consisting of systolic congestive heart failure (sCHF), pulmonary edema (PE), cardiogenic shock (CS), vasodilative shock (VS) and normal state;
(b) means for determining the range of Cpᵢ and SVRᵢ paired values corresponding to each of said hemodynamic states;
(c) means for determining the Cpᵢ and SVRᵢ paired value of said subject;
(d) means for comparing the Cpᵢ and SVRᵢ paired value of said subject to the ranges of Cpᵢ and SVRᵢ paired values determined by the means of (b); and
(e) means for determining the range of Cpᵢ and SVRᵢ paired values which is most similar to the Cpᵢ and SVRᵢ paired value of said subject, the hemodynamic state corresponding to said range indicating the hemodynamic state of said subject.

13. An apparatus according to claim 12 wherein said means is a computer.

14. An apparatus according to claim 12 wherein said Cpᵢ and SVRᵢ paired values are plotted on a graph and said range of Cpᵢ and SVRᵢ paired values indicative of each of said hemodynamic states is indicated by a delineated area on said graph.

15. An apparatus according to claim 14 wherein said graph is substantially equivalent to FIG. 5.

16. An apparatus according to claim 12 wherein said ranges of Cpᵢ and SVRᵢ paired values indicative of each of said hemodynamic states are calculated by statistical analysis and said Cpᵢ and SVRᵢ values of said subject are compared to said ranges by a statistical method.

17. An apparatus according to claim 16 wherein said range of Cpᵢ and SVRᵢ paired values indicative of each of said hemodynamic states is displayed in a graph format on a display screen.

18. An apparatus according to claim 12 wherein Cpᵢ is calculated from the product of the cardiac index (CI) and the mean arterial blood pressure (MAP).

19. An apparatus according to claim 18 wherein said cardiac index and/or said blood pressure is measured by an invasive measuring technique.

20. An apparatus according to claim 19 wherein said measuring technique for measuring the cardiac output employs a Swan-Ganz catheter.

21. An apparatus according to claim 12 wherein cardiac output and/or said blood pressure is measured by a non-invasive measuring technique.

22. An apparatus according to claim 12 for monitoring the hemodynamic state of a subject, comprising means for following the change in position of the paired Cpᵢ and SVRᵢ values of the patient with respect to the predetermined set of values over a predetermined period of time, thereby monitoring the hemodynamic state of said subject over said period of time.
